(19) United States
(12) Patent Application Publication
(10) Pub. No.: US 2021/0166816 A1
(43) Pub. Date: Jun. 3, 2021

(54) DYNAMIC CAREGIVER SUPPORT

(71) Applicant: International Business Machines Corporation, Armonk, NY (US)

(72) Inventors: Naweed A. Khan, Johannesburg (ZA); Oliver Bent, Oxford (GB); Aisha Walcott, Nairobi (KE); Komminist Weldemariam, Ottawa (CA)

(21) Appl. No.: 16/699,088

(22) Filed: Nov. 28, 2019

(51) Int. Cl. G16H 50/30; G16H 10/60; G16H 20/60; G16H 20/10; G06Q 10/10

(52) U.S. Cl. CPC G16H 50/30; G16H 10/60; G06Q 10/1093; G16H 20/10; G16H 20/60

(57) ABSTRACT

A method of operating a support system includes enabling electronic communications with at least one senor of user conditions and at least one database of user activity, collecting, via the electronic communications, a plurality of data points from the sensor and the database, determining a context of the user, calculating at least one affinity score of the user for each of at least one patient, predicting a risk of passive illness associated with the user, identifying, using the risk of passive illness, an amelioration action, and outputting a signal reducing the risk of the passive illness.

The present invention relates to the electronic arts, and more particularly to a system and method for monitoring and correcting the status of an entity.

Generally, caregivers provide medical intervention support to a patient. A caregiver may be any entity associated with a patient's care. In the context of patient care, it is recommended that caregivers take a proactive approach to selfcare, e.g., before entering the ward, interacting with a patient, etc. In many cases the caregivers tend to ignore or are unaware of these recommendations, which increases the risk of sickness, infection, and changes in mental conditions (e.g., anxiety, depression, fatigue, sleep problem, etc.). Furthermore, with the caregiver's focus trained on the patient, the caregiver is likely to transition into a state of passive illness where the caregiver is unaware of deleterious conditions that do arise.

### BRIEF SUMMARY

According to one or more embodiments of the present invention, a dynamic online and offline Caregiver Support System (CSS) for effective patient outcomes, using various affinity measures within a caregiver (social) network. The invention also provides dynamic online and offline notification of availability of caregiver support by an intelligent module within the caregiver network towards calendars, historic medical record data, and other contextual artefacts.

According to one or more embodiments, a method of operating a support system includes enabling electronic communications with at least one senor of user conditions and at least one database of user activity, collecting, via the electronic communications, a plurality of data points from the sensor and the database, determining a context of the user, calculating at least one affinity score of the user for each of at least one patient, predicting a risk of passive illness associated with the user, identifying, using the risk of passive illness, an amelioration action, and outputting a signal reducing the risk of the passive illness.

As used herein, “facilitating” an action includes performing the action, making the action easier, helping to carry the action out, or causing the action to be performed. Thus, by way of example and not limitation, instructions executing on one processor might facilitate an action carried out by instructions executing on a remote processor, by sending appropriate data or commands to cause or aid the action to be performed, by displaying the action and associated commands on one or more user computing devices (e.g., mobile phone, smartwatch, Augmented Reality (AR) display glasses, etc.). For the avoidance of doubt, where an actor facilitates an action by other than performing the action (e.g., by causing the action to be performed), a related action may nevertheless be performed by some entity or combination of entities.

One or more embodiments of the invention or elements thereof can be implemented in the form of a computer program product including a computer readable storage medium with computer usable program code for performing the method steps indicated. Furthermore, one or more embodiments of the invention or elements thereof can be implemented in the form of a system (or apparatus) including a memory, and at least one processor that is coupled to the memory and operative to perform exemplary method steps. Yet further, in another aspect, one or more embodiments of the invention or elements thereof can be implemented in the form of means for carrying out one or more of the method steps described herein; the means can include (i) hardware module(s), (ii) software module(s) stored in a computer readable storage medium (or multiple such media) and implemented on a hardware processor, or (iii) a combination of (i) and (ii); any of (i)-(iii) implement the specific techniques set forth herein.

Techniques of the present invention can provide substantial beneficial technical effects. For example, one or more embodiments may provide for:

an adaptive caregiver guidance derived from work-tasks and environmental data, including executable actions facilitating rehabilitation, thereby improving an outcome for a caregiver (including reducing risks thereof);

early identification of indicators of passive illness in the caregiver;

estimating an impact of missing out one or more scheduled caregiver events due to the identified indicators of passive illness in the caregiver;

identification of a set of amelioration actions reducing the occurrence of passive illness based on the estimated impacts;

ergonomic data collection, processing and system interactivity considerations optimized according to a set of sensor signals available within a work-task environment; and

ergonomic data collection, processing and system interactivity considerations adjusted according to conditions.

These and other features and advantages of the present invention will become apparent from the following detailed description of illustrative embodiments thereof, which is to be read in connection with the accompanying drawings.

### BRIEF DESCRIPTION OF THE SEVERAL VIEWS OF THE DRAWINGS

Preferred embodiments of the present invention will be described below in more detail, with reference to the accompanying drawings:

FIG. 1 is a diagram of a support system configured to predict passive illness according to an embodiment of the present invention;

FIG. 2 is a diagram of a method for predicting passive illness and its intervention according to an embodiment of the present invention;

FIG. 3 is a diagram of a method for detecting and storing emotional and cognitive trajectories using existing methods such as facial feature classification and tone analysis according to an embodiment of the present invention;

FIG. 4 is a diagram of a method for passive illness prediction according to an embodiment of the present invention; and

FIG. 5 is a block diagram depicting an exemplary computer system embodying a method of operating a support system monitoring and correcting the status of an entity, according to an exemplary embodiment of the present invention.

### DETAILED DESCRIPTION

According to one or more embodiments of the present invention, a dynamic online and offline Caregiver Support System (CSS) for effective patient outcomes, using various affinity measures within a caregiver (social) network. The invention also provides dynamic online and offline notification of availability of caregiver support by an intelligent module within the caregiver network towards calendars, historic medical record data, and other contextual artefacts.

According to one or more embodiments of the present invention, a CSS monitor is associated with a caregiver. It can be assumed that the caregiver is associated with one or more patients. Data about patient conditions (including types, conditions, context, and duration of the illness/disease, etc.) is determined by, and/or, provided to the CSS monitor, which identifies one or more affinity scores of the caregiver (to each patient), predicts the occurrence of passive illness indicators associated with the caregiver, and identifies a set of amelioration actions to minimize the occurrence of passive illness of the caregiver.

According to one or more embodiments of the present invention, the CSS monitor estimates an impact of missing out one or more (caregiver) events (e.g., scheduled interactions/events) due to the predicted occurrence of passive illness. In the context of caregiving, the scheduled interactions/events can include, for example, client calls/meetings, flight/travel, and appointments, such as sitting for an exam, road driving license test, etc.

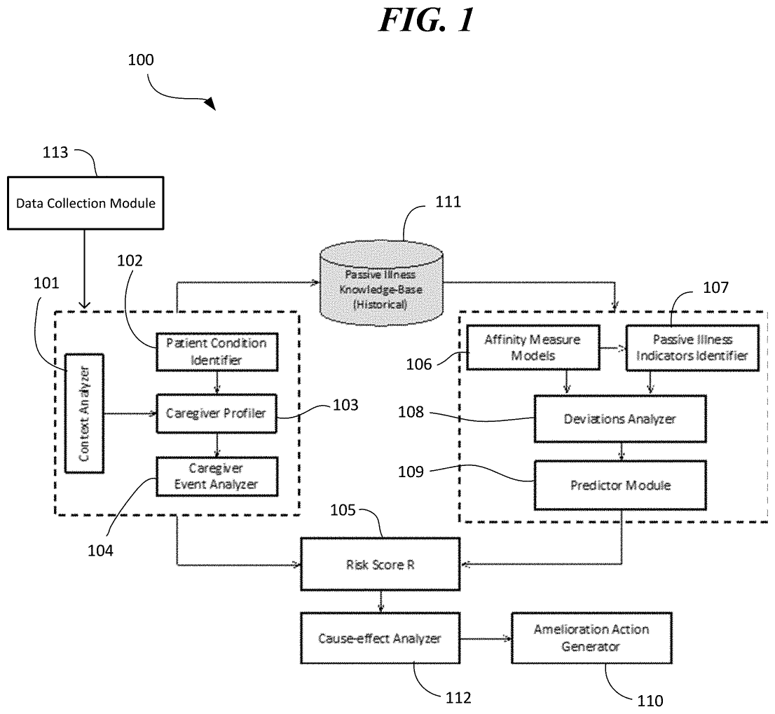

FIG. 1 illustrates a CSS monitor 100. The CSS monitor 100 is associated with at least one caregiver. The CSS monitor 100 comprises a context analyzer 101, a caregiver profiler 103, a caregiver event analyzer 104, a risk score evaluator 105, affinity measure models 106 (e.g., database), passive illness indicator identifier 107, a deviations analyzer 108, a prediction module 109, amelioration action generator 110, a database 111 of patient and caregiver data, and a data collection module 113. According to at least one embodiment, the CSS optionally includes a patient condition identifier 102.

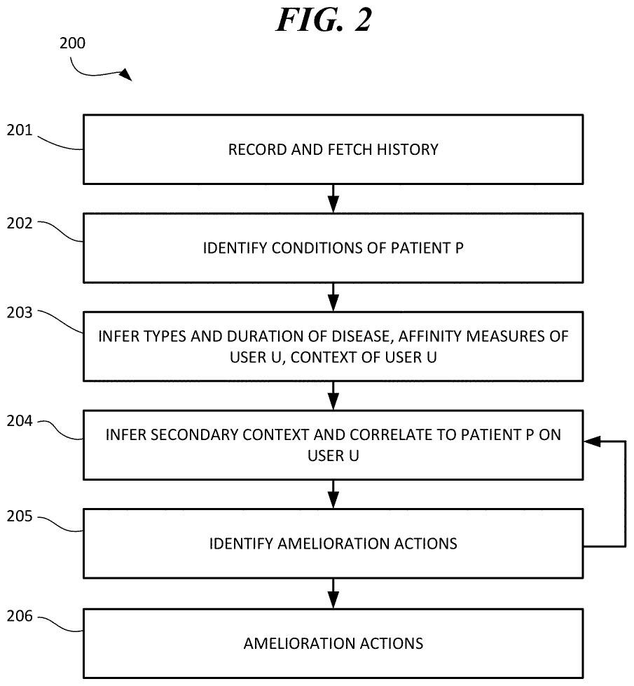

According to one or more embodiments, the CSS monitor 100 comprises a cause-effect analyzer 112, which identifies positive/negative side effects, correlating patient-user medical similarity, sequencing pattern history, etc. based on deploying a series of cognitive tools and algorithms. According to at least one embodiment, these causes and effects are used to determine a set of ameliorative actions. According to at least one embodiment, a set of recommended amelioration actions are optimized by the cause-effect analyzer 112, which employs reinforcement learning as feedback for continual learning of effects and assigning the reward function (positive/negative). Reinforcement learning maps states to actions by exploration, where the mapped action has a highest cumulative reward. More particularly, according to some embodiments, the cause-effect analyzer 112 determines one or more ameliorative actions (see also, FIG. 2, block 205) by identifying at least one positive/negative side effect (e.g., reinforcement rewards) from acting on the set of amelioration actions generated in 110, the correlating/measuring affinity patient(s)-caregiver medical similarity, patient(s)-caregiver interaction pattern and history, etc., based on deploying one or more machine learning models (e.g., k-means, decision tree, deep learning algorithms, etc.).

According to some embodiments, reinforcement rewards are defined by the feature set, e.g., patient(s)-caregiver medical similarity, patient(s)-caregiver interaction pattern and history, etc., and are scored by the affinity measure model module 106, wherein the cognitive tool used in one embodiment is defined using, for example, an unsupervised feature modelling, a dimensionality reduction and nearest neighbor technique to cluster hyperplane correlations of similar features, joint and individual, shared across contexts.

According to some embodiments, predicting a relationship of user with the patient is based on any multi-class prediction algorithm, in particular the application of a decision-tree learned from the detected features (interactions, times and proximities) and an associated relationship(s). The affinity score may then be built up based on cohort analysis of the predicted relationships. With an associated affinity score [0,1] describing a low to high coupling or interaction between the user and patient given their relationship and a comparison(s) with other user/patient interactions, times and proximities across relationship cohorts. The affinity measure model module 106 thereby provides index and distance metrics that rank the contextual similarity of features to one another. In one or more other embodiments, affinity measure model is a cosine similarity algorithm that measures a cosine of an angle between two feature sets (represented by vectors) projected in a multi-dimensional space. Thus, the similarity (i.e., affinity) between A and B can be calculated as:

$$similarity = cos ( θ ) = \frac{\vec{a} \cdot \vec{b}}{‖ \vec{a} ‖ \cdot ‖ \vec{b} ‖}$$

where \vec{a} and \vec{b} are the vectors of A and B respectively.

According to some embodiments, the affinity measure model can be implemented using Pearson's correlation or correlation similarity (measuring how much two feature vectors are correlated, where a higher correlation indicating a higher degree of similarity), a Mean Squared Difference (MSD) (finding an average squared divergence between the two vectors—MSD tends to put more weight into penalizing larger errors—), etc.

According to some embodiments, the cause-effect analyzer 112 improves the determination of the amelioration action(s) by employing a reinforcement learning algorithm, which uses feedback for continual learning of the effects of different available amelioration actions. According to at least one embodiment, the cause-effect analyzer 112 assigns a reinforcement learning reward (positive/negative) to each amelioration action.

According to some embodiment, the cause-effect analyzer 112 outputs the one or more amelioration actions with rewards, as defined by the amelioration action generator 110 (see also, FIG. 2, block 205). According to embodiments of the present invention, the output can be an electronic communication (e.g., a signal, notification message, an alert) to the caregiver's mobile device (including to an appropriate health tracking application executing on the mobile device), electronic calendar, etc. According to at least one embodiment, the electronic communication causes an outdate in the caregiver's electronic calendar, rescheduling an appointment with the patient, scheduling coverage for the patient by another caregiver, arranging consultation with care providers, provide educational materials, recommending a caregiver to take a physical exercise, etc. According to some embodiments, the automated scheduling can attempt to optimize outcomes/experiences for all stakeholders, including the caregiver(s) and the patient.

The CSS monitor 100 creates a passive illness knowledge-base 111. Using a plurality of data sources, caregiver data, other contextual data sources and disease corpuses (e.g., PubMed—a publicly available resource for disease name recognition and concept normalization), generally referred to at the data collection module 113, a corpus of cross-referenced passive illness knowledge-base is created and updated within the passive illness knowledge-base 111. According to at least one embodiment, optionally, patient data is inferred and/or detected by the patient condition identifier 102 to be used to profile a caregiver by the caregiver profiler 103. The patient data may include current and historical medical conditions and complications, profiles, real-time periodic physiological data (e.g., one or more physiological parameters, activity data, food and medicine intake data, and/or sleep and rest data, associated with the patient) received from the one or more devices/sensors (e.g., camera, biometric and IoT feed such as smartwatch, etc.), etc. Optionally, non-medical patient data and patient contextual data is securely stored as the patient profile in 102 (e.g., age, gender, race, number of family members, occupation, address, etc.). The caregiver data may include contextual data of each of a plurality of caregivers associated with the patient received from one or more data sources, caregiver historical medical conditions, profile and context of caregiver (e.g., age, gender, skill, education, job, prior experience, awareness, etc.). The other contextual data sources may include profile of a care location, inferred crowd density/information, and so on.

According to one or more embodiments, the data collection module 113 includes sensors (e.g., data sources), network connections to databases (e.g., including the knowledge-base 111), etc.

FIG. 2 is a diagram of a method 200 for predicting passive illness and providing intervention support. Using opt-in approaches, patent histories are recorded and/or fetched from an appropriate database 201. At block 202, patient conditions are identified. At block 203, the type(s), condition(s), and duration of the illness/disease are inferred, together with affinity measures of the caregiver (i.e., user U) and a context of the caregiver. At block 204, secondary context can be inferred (e.g., patterns and characteristics of illness of the patient, patient reactions to treatment, etc.). Further, at block 204, any progress of the patient can be estimated, an affinity of the patient with respect to one or more physiological parameters, activity data, food and medicine intake, and/or sleep and rest patterns, etc., can be determined. Amelioration actions are identified at block 205 and output to the caregiver (e.g., a caregiver's mobile device) at block 206.

In one exemplary implementation, the CSS identifies that a first caregiver's patient has been sick for 11 days, the CSS learns that the first caregiver's patient stayed 5 days at a hospital, 4 days at home and the last 2 days in another location). The CSS further learns that the first caregiver has visited the patient 11 times (while the patient was at hospital or at home) and a second caregiver has visited the patient over 25 times within the 9 days and interacted with the patient over 47 times. From the interactions and visits data, the CSS detects that the second caregiver is showing low energy, poor sleeping, and light sneezing and coughing during nighttime. The CSS also accesses (up on a proper consent granted, as needed) calendar entries, which show that the second caregiver has a pre-scheduled driving license test (specifically road test) for a certain date and location, and that the first caregiver will be traveling for client meeting. The CSS predicts the future occurrence of possible passive illness conditions (indication of a cold) for the second caregiver. For example, based on an analysis of historical data by the CSS, it may be known that, if the second caregiver contracts a cold, it will take at least a week to recover. The CSS performs a risk analysis and determines a high chance (e.g., 92%) of the second caregiver missing the appointment (i.e., driving exam), again based on the analysis of historical data, which will lead to the expiration of the second caregiver's license if the second caregiver falls sick in 2 days (in the example, the second caregiver cannot re-schedule the test, which can be inferred from a test administer entity). Historical health data of the first caregiver shows patterns of contracting colds from the second caregiver (approximately 3 days later). The CSS then generates personalized recommendations to both the first and the second caregivers.

According to one or more embodiments of the present invention, the CSS determines a care location context from one or more data sources specific of the location. The CSS profiles a location or patterns of locations where a patient has received care (e.g., home, clinic, hospital, etc.) as well as profiling various caregivers. The CSS is configured to infer crowd density/information including the number of people around the patient at a given time, by employing one or more machine learning algorithms such as activity detection models, with or without computer vision-based tracking models (e.g., GPS logs, speaker voice-analysis on mobile phone, wearable devices, etc.). Further, according to at least one embodiment, care location profiles and context (e.g., crowd density analysis) are inferred from local communication devices (e.g., beacons, WiFi access point, surveillance cameras, information servers, stream firewalls). This is done by the Context Analyzer module 101.

Via opt-in approach, data from social media of the caregiver can be used to create a profile of the current state of mind of the caregiver. According to some embodiments, the CSS tracks sleeping pattern data from wearable devices, which can be used to enhance the profile of the state of mind of the caregiver. For example, a restless night can indicate stress or anxiety, which will be part of the profile of the mental state of the caregiver.

Referring to measures of affinity values of a caregiver (affinity measure model module 106), these affinity values/scores include caregiver-patient interaction, caregiver-medication interaction, caregiver-meal interactions, caregiver-caregiver interaction (of different patients), etc. In one exemplary embodiment, the caregiver-patient affinity value/score is calculated by looking at explicit and implicit interactions that caregivers involved with and actions that caregivers taken, and factoring in 1) the current and future frequency of interactions, 2) time spend at each interaction with patient, 3) how close (proximity) the caregiver to the patient or cohort of patients at each interaction, 4) what actions the caregiver performed, and 5) the relationship of caregiver with the patient.

According to some embodiments, the affinity scoring performed by the affinity measure model module 106 is calculated using unsupervised feature modelling, dimensionality reduction and nearest neighbor techniques cluster hyperplane correlations of similar features shared across caregivers. Thereby providing index and distance metrics that rank caregivers by contextual similarity to one another.

According to one or more embodiments of the present invention, the computed affinities are used to measure similarity of caregivers (cohort analysis). This similarity measure can be constructed by a probabilistic model (e.g., using Markov model process). IBM's Contact Tracer device and system can be used to identify patient-caregiver and caregiver-caregiver interactions within the caregiving context and used to anchor the emotional or cognitive states and/or trajectories to interactions (see below). In one embodiment, a collective affinity of a caregiver to a patient is computed based on the type/number of engagements between the entities. For example, a collective affinity can increase with the number of times caregiver U interacts with patient P (e.g., during mealtime, for administering medication, etc.).

According to one or more embodiments of the present invention, the CSS infers the caregiver's emotional trajectories from the affinity values/scores—including inferring from speech, biometric sensors, facial expression analysis, caregiver typing, browsing or viewing speed, etc. The CSS then determines the caregiver condition and context in relation to the patient or cohort of patients. This further includes learning the caregiver cognitive state trajectory. Techniques in the prior art identifying emotional or cognitive states based on a set of predefined inputs (facial features, speech tone analysis, biometric sensors, etc.) may be employed to classify emotional state and then infer the caregiver cognitive state trajectory. The identification of a set of emotional indicators for the caregiver is based on a standard emotional classification such as: Anger, Disgust, Fear, Happiness, Sadness, Surprise, Neutral. By using these features and augmenting the classification with additional sources (e.g., sensor clusters) from a data collection module, a comprehensive feature set from external sensors can provide the context of states.

According to one or more embodiments of the present invention, the CSS determines one or more contextual factors associated with the caregiver based on analyzing frequency of caregiver-patient interactions, caregiver cognitive state trajectory, profile of each care facility, relative relation of caregiving affect to professional caregivers, and so on.

According to one or more embodiments of the present invention, the CSS predicts the occurrence of a passive illness condition (Predictor Module 109). In one embodiment, predicting occurrence of a passive illness condition in a caregiver or cohort of caregivers under consideration using a neural network model, with weighted connections, an input and an output, the said weighted connections resulting from training said neural network model. According to one or more embodiments of the present invention, the input to the neural network model comprises the computed affinity scores, caregiver historical medical conditions, profile and context of caregiver (e.g., demographics, education, job, prior experience, aware-ness, etc.), contextual data (e.g., profiles of care facilities/locations, inferred crowd densities), and so on. The input is configured to receive data for the caregiver consideration and, based on said weighted connections, neural network model is configured to provide at said output one or more passive illness indicators indicating the likelihood or risk of occurrence of the passive illness condition in said caregiver.

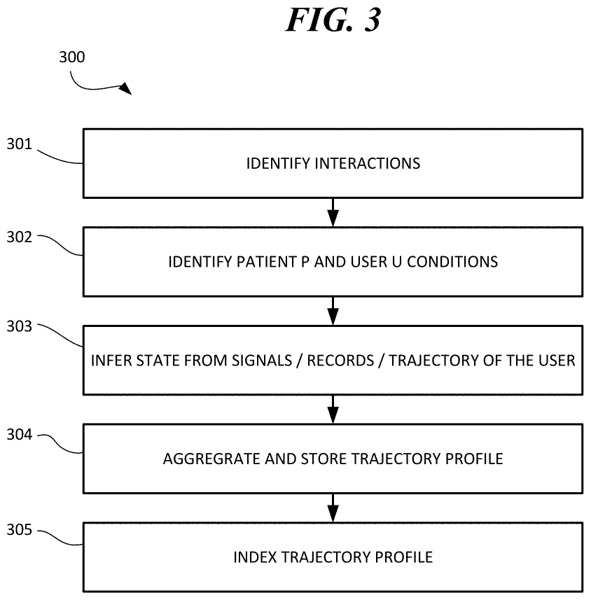

FIG. 3 is a flow diagram of a method 300 for detecting and storing emotional and cognitive trajectories according to some embodiments. According to at least one embodiment, emotional and cognitive trajectories of the caregiver can be determined using facial feature classification and tonal analysis (e.g., using pretrained deep learning models). More particularly, according to an embodiment of the present invention, the interactions are used to anchor the emotional or cognitive states and/or trajectories to interactions 301. These interactions can include patient-user and user-user interactions within the caregiving context.

According to an one or more embodiments, a neural network is trained and deployed to output one or more passive illness indicators 302. The neural network model detects emotion and cognitive trajectories of the user associated with the patient and illness, predicts and/or detects fever/headache, predicts and/or detects changes in skin blood perfusion and oxygenation, inferring user depression/anxiety from trajectory emotion, identifying changes in speech/voice, cognitive state, affective state, interaction with medication, etc.

According to an one or more embodiments, one or more events associated with the user can be identified and/or predicted 303. For example, an event associated with the user (i.e., the caregiver) can be identified from an electronic calendar of the user and/or other data sources such as text or audio conversational data. An example of an event includes scheduled client calls/meetings, upcoming flight/travel, upcoming appointments (e.g., sitting for exam, a driver's license test), etc., that require the user's attention.

According to some embodiments, the trajectory profile of the user (e.g., the profile over time) is stored in a database 304. According to at least one embodiment, data from different trajectory profiles can be aggregated 304 and analyzed to predict risk of passive illness, etc.

According to at least one embodiment, the trajectory profile is indexed 305 to provide an efficient database lookup for the trajectories of similar passive illness profiles. This operation allows for the entire trajectory, or subsets within predetermined interaction windows (e.g., time-stamped beginning and ending of a patient-caregiver interaction), to be encoded by a feature vector. The feature vectors thus provide a summary of the aggregated trajectory as a function of the interaction window and are thereafter compared for determining passive illness risks aggregated across similar caregiver-patient profiles.

In one embodiment, the impact of missing out on one or more events due to the predicted occurrence of passive illness is estimated. For example, the impact of missing an event can be measured based on estimating an ability of the user to carry out the one or more identified, or predicted, events based on an analysis of historical events. According to one embodiment, based on the estimated ability, and through the analysis of the identified events, a risk score R is computed that indicates the estimated impact of the user missing the one or more events.

According to some embodiments, the CSS observes the state and action pairs associated with (e.g., normal) caregiving for professional and non-professional caregivers known not to be experiencing a passive illness. Through these observations the CSS learns a reward functions for the act of caregiving using inverse reinforcement learning. The CSS learns through demonstration and outputs recommendations (i.e., amelioration actions) to a user (e.g., a caregiver who is struggling while also providing an understanding of behavior (or policy) under normal caregiving). If the user's actions deviate from a normal behavior, then the user is increasing a risk of passive illness.

In one embodiment, the CSS builds a caregiver health graph network (HGN) where nodes indicate the health of individual users and edges indicate a risk of passive disease transmission calculated from affinity measures. By using subset scanning techniques to determine how the health condition of surrounding people is changing over a period of a time.

Briefly, a subset scanning technique or method is a variation of statistical machine learning method suited to identify anomalous patterns in real-world data by characterizing and understanding where, when, and how observed phenomena (e.g., of systems) deviate from their expected dynamics. More specifically, anomalous pattern detection includes a task of identifying subsets of data points that systematically differ from the underlying model. In the spatiotemporal domain, timely identification of such patterns can allow for effective interventions. In at least one embodiment, the CSS 100 uses a variation of the subset scanning technique to identify, detect and characterize anomalous group of connected caregivers in the health graph network. The quantified anomalousness score may indicate an increased risk of passive illness for a caregiver. From the HGN, a subset scanning technique is used to detect anomalous subgroups of connected caregivers in one embodiment. A subgroup can be identified, e.g., based on their connections, and compared with another subgroup.

Exemplary implementations of the CSS 100 using a variation of the subset scanning technique can include:
a. A HGN having health information of caregivers securely stored at graph nodes (e.g., perceived health level, biometric information, illness type, symptoms, duration of illness, duration of symptoms);
b. A HGN having a plurality of interaction information securely stored at the graph edges (e.g., frequency, type and duration of interaction);
c. Interaction event modeling triggered by IBM's Contact Tracer devices that record graph edge interactivity and identify anomalies interactivity;
d. Estimated health conditions modeled via a graph-based machine learning approach, predicting future interactivity and caregiver features from historical data. The estimation and comparison of historically predicted features can be compared to current caregiver healthcare conditions, providing feedback features to optimize against;
e. Predicted features identifying anomalous graph subsets that may transmit passive illnesses to one another.

Through the analysis of the occurrence of the passive illness conditions with identified caregiver important events (causal-effect analysis), system determining one or more amelioration actions to minimize the predicted occurrence of the passive illness.

The method of amelioration actions generator further takes the estimated fitness of the caregiver to the identified caregiver events (e.g., client calls, travel event, appointments, etc. that requires the caregiver presence).

According to one or more embodiments of the present invention, depending upon a risk threshold of a multi-level classifier (e.g., employing voting ensemble learning), the amelioration action generator 110 recommends or outputs a set of amelioration actions or interventions (Y), which can include:
recommending mindfulness activities such as deep breathing;
recommending a user to stay away from a care for duration time t;
recommending a user to wear protective cover during a certain time of the day;
reminders to take medication;
arranging consultation with care providers, provide educational materials;
scheduling coverage for the patient by another caregiver;
etc.

In one exemplary scenario, a reinforcement learning model is trained to learn to recommend one or more the interventions (e.g., deep breathing), and to monitor if the caregiver performs the recommended intervention (e.g., deep breathing) or ignores the recommended intervention. In this example, the system assigns a positive reward if the caregiver performs the deep breathing and zero otherwise. The set of ameliorative actions to be taken can determined by compiling a knowledge corpus of passive illnesses, a corpus of amelioration actions, sets of preventative measures and active steps to treat ailments and symptoms. The corpus can be compiled from hierarchical trust sources, such as specialists, professional doctors, rated forum posts, general online information, user actions collected from the user devices over a period of time, etc.

In one exemplary embodiment, the recommendation process is modeled as a sequential decision making problem, in which a recommender (e.g., a computer system configured to performed one or more methods described herein) interacts with caregivers (e.g., the environment) to suggest a list of amelioration actions sequentially over a plurality of timesteps, while attempting to maximize the cumulative rewards of the whole recommendation process. According to some embodiments, the recommendation process is modelled by a Markov Decision Process (MDP), (S,A,P,R,γ)—(States (S), Actions (A), Transitions (P), Reward (R), Discount Rate (γ))—.

In an exemplary MDP, a state, s, is a representation of caregiver's positive interaction history with recommender, as well as his profile and context information. Further, an action, a, is a continuous parameter vector denoted as a ∈ R^{1×k} (where k is a variable of the parameterized action space). Each intervention it ∈ R^{1×k} has a ranking score, which is defined as the inner product of the action and the intervention embedding, i.e., it a T, where the top ranked ones (e.g., determined by threshold) are recommended. Moreover, referring to the transition, P, a transition between states after taking action a is modeled as the representation of caretaker's positive interaction history. Hence, once the user's feedback is collected, the state transition is determined. Referring now to the reward, R, given the intervention based on the action a and the caretaker state s, the caretaker provides feedback, e.g., indicating that the intervention (e.g., deep breathing) has been taken, indicating that the intervention has not been performed, etc. The recommender receives an immediate reward R (s, a) according to the caregiver's feedback. The discount rate, γ ∈ [0,1], is a factor measuring the present value of long-term rewards. In the case of γ=0, the recommender considers only immediate rewards, and long-term rewards are ignored. On the other hand, when γ=1, the recommender treats immediate rewards and long-term rewards as equally important.

Considering the current caregiver state and immediate reward to the previous action, the recommender takes an action. According to some embodiments, an action corresponds to neither recommending an intervention, nor recommending a list of interventions. Instead, an action is a continuous parameter vector. Taking such an action, the parameter vector is used to determine the ranking scores of all the candidate interventions, by performing inner product with intervention embeddings. According to some embodiments, all the candidate interventions are ranked according to the computed scores and top-M suitable interventions are recommended to the caregiver. Taking the recommendation from the recommender, the caregiver provides feedback to the recommender (e.g., either explicitly through answering a question provided through an application interface or through mining data about the caregiver's activity that the caregiver makes available to the system) and the caregiver state is updated accordingly. The recommender receives rewards according to the caregiver's feedback. Without loss of generality, a recommendation process is a T timestep trajectory can be written as (s₀, a₀, r₀, s₁, a₁, r₁, . . . ,s_{T-1}, a_{T-1}, r_{T-1}, s_{T}).

According to at least one embodiment, a reinforcement used for recommender-caregiver interactions in MDP can be implemented by the amelioration action generator 110.

According to one embodiment, the recommended sequence of actions or interventions is output by the reinforcement learning model. Observing for each caregiver particular personalized sequences of actions, which may be optimal in the tasks of both returning/or maintaining the caregiver in a healthy state.

According to one or more embodiments of the present invention, the set or sequence of amelioration actions are presented to the caregiver/user using existing notification services, such as smart device prompts, virtual assistant reminders, chatbot communication from instant messaging services that users are active participants in, etc.

According to one or more embodiments, presenting the set or sequence of amelioration actions to the caregiver includes displaying the action and associated commands on one or more caregiver/user computing devices such as mobile phone, smartwatch, Augment Reality (AR) display glasses, etc.

According to one or more embodiments of the present invention, the amelioration action generator 110 uses voting ensemble learning (i.e., collection of different models working together on a single set as an ensemble, and using a voting classifier to select a final output, e.g., by majority voting or as an average of two or more of the different models) to determine a cascading set of actions associated with passive illnesses progressions and presents the recommendation to the user based on severity and knowledge of previous steps taken. The CSS applies a feedback cycle from recommendations, identifies steps acted upon, measures changes in cognitive states and trajectories in order to learn through reinforcement from action, state pairs.

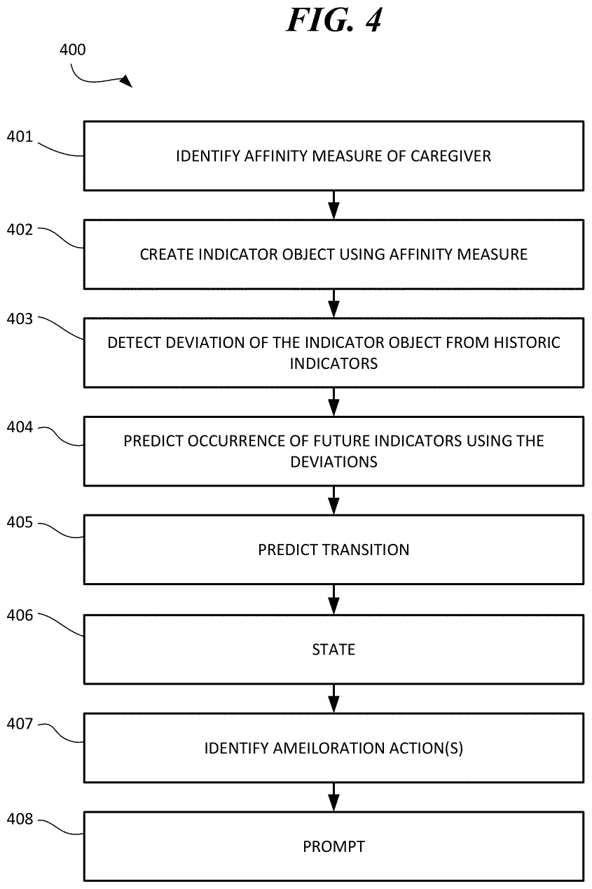

FIG. 4 is a diagram of a method 400 shows flowchart illustrating how the present invention can be implemented. According to at least one embodiment of the present invention, the CSS system maps caregiver healthcare conditions and interactions (retrieved from a HGN), and uses an Affinity Measure Models module 106 to identify similar caregivers at block 401. According to some embodiments, supervised machine learning models are trained to predict a likelihood of passive illnesses occurring using the HGN and the passive illness indicators identifier 107. A deviations analyzer 108 detects deviations of the caretaker's indicators from historic values at block 403 and a predictor module 109 predicts the occurrence of future indicators based on the deviations at block 404. According to at least one embodiment, the predictor module 109 further predicts a transition to passive illness at block 405 and a caretaker state at block 406. Identified caregiver features and symptoms, such that correlations (e.g., high correlations) between affinity measures at block 402 provide a feature subset that is indicative of possible high-risk factors (see FIG. 1, 105) that may contribute to the identification of the passive illness through the functions of one or more of blocks 403-406. According to at least one embodiment, the amelioration action is identified at block 407 (i.e., by the amelioration action generator 110) and presented to the caregiver at block 408 to facilitate an action. Here, presentation can include a notification on a mobile device, an email, calendar event, etc. That is, the amelioration action

According to at least one embodiment, optionally, privacy is handled by the data collection module 113, by capturing information from a subset of users (e.g., nurses, professional caregivers, etc.) as part of their daily work routine (e.g., in a hospital, clinic, clinical trial, the patients home, etc.), where information gathered is clearly outlined as part of contractual obligations. Contact tracer systems can be used here, to anchor patient and caregiver interactivity to an HGN, providing better contextual information. The storing and interpretation of this information is subsequently used to train the machine learning models. Home caregivers may thereafter opt-in to have their information parsed through these models, where transfer learning and domain adaptation techniques from few-shot learning may be applied, predicting the likelihood of contracting passive illnesses, by storing a sparse set of user information or without storing the information by applying models at inference time.

### Other Embodiments

In one embodiment, a risk score is determined using the patient's illnesses and the corresponding time frame for which the illness is infectious. The risk of passive illness is a value [0,1] output from a neural network which takes as inputs the sequences of the emotional, cognitive and health trajectories.

We also use the estimated time for symptoms to appear for a specific illness/disease to determine the recommendations, for some symptoms show up very early when the patient is infected, other illnesses show up a few days later after infection. Early detection and treatment for some diseases can reduce the severity of the infection. This is combined in the Amelioration Action Generator.

Recapitulation:

Referring to FIG. 4, according to at least one embodiment of the present invention, a possible sequence of events identified by the system is highlighted by way of example:
a. Caregiver healthcare conditions and interactions are mapped via an HGN
b. Affinity measure models 106 identify similar caregivers 401.
c. Supervised machine learning models are trained to predict the likelihood of passive illnesses occurring using the HGN 107.
d. Deviations 108 from the predicted 109 caregiver features and symptoms are identified, such that the highest correlations between affinity measures 402 provides a feature subset that is indicative of possible high-risk factors 105 that may contribute to the identification of the passive illness.
e. The set of ameliorations are identified 407 and presented 408 to the caregiver to facilitate action 110.

The methodologies of embodiments of the disclosure may be particularly well-suited for use in an electronic device or alternative system. Accordingly, embodiments of the present invention may take the form of an entirely hardware embodiment or an embodiment combining software and hardware aspects that may all generally be referred to herein as a “processor,” “circuit,” “module” or “system.”

Furthermore, it should be noted that any of the methods described herein can include an additional step of providing a computer system implementing a method for anomaly alarm consolidation. Further, a computer program product can include a tangible computer-readable recordable storage medium with code adapted to be executed to carry out one or more method steps described herein, including the provision of the system with the distinct software modules.

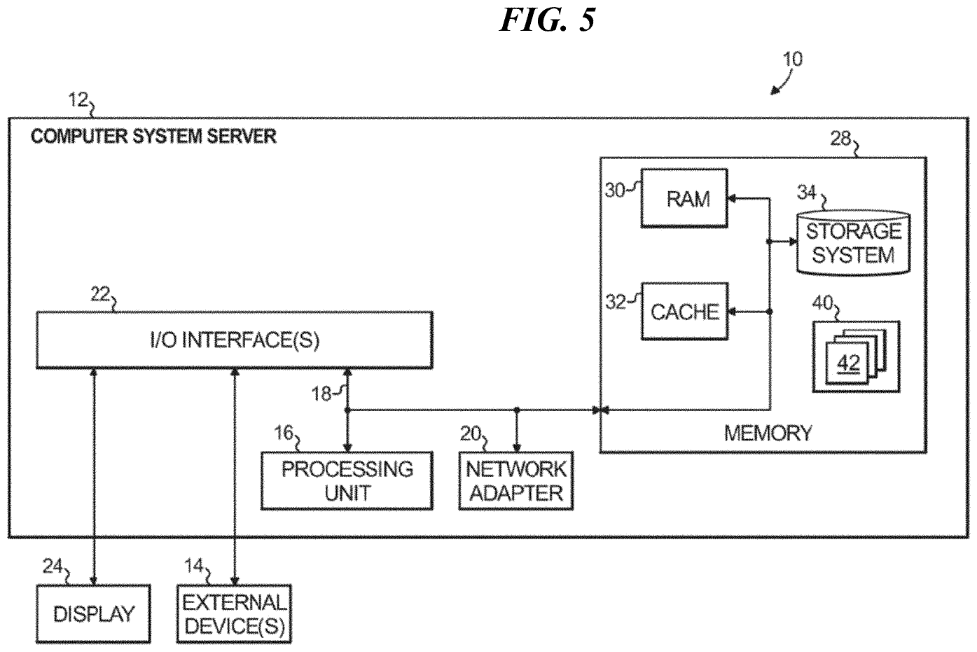

One or more embodiments of the invention, or elements thereof, can be implemented in the form of an apparatus including a memory and at least one processor that is coupled to the memory and operative to perform exemplary method steps. FIG. 5 depicts a computer system that may be useful in implementing one or more aspects and/or elements of the invention, also representative of a cloud computing node according to an embodiment of the present invention. Referring now to FIG. 5, cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove.

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 5, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, and external disk drive arrays, RAID systems, tape drives, and data archival storage systems, etc.

Thus, one or more embodiments can make use of software running on a general purpose computer or workstation. With reference to FIG. 5, such an implementation might employ, for example, a processor 16, a memory 28, and an input/output interface 22 to a display 24 and external device(s) 14 such as a keyboard, a pointing device, or the like. The term “processor” as used herein is intended to include any processing device, such as, for example, one that includes a CPU (central processing unit) and/or other forms of processing circuitry. Further, the term “processor” may refer to more than one individual processor. The term “memory” is intended to include memory associated with a processor or CPU, such as, for example, RAM (random access memory) 30, ROM (read only memory), a fixed memory device (for example, hard drive 34), a removable memory device (for example, diskette), a flash memory and the like. In addition, the phrase “input/output interface” as used herein, is intended to contemplate an interface to, for example, one or more mechanisms for inputting data to the processing unit (for example, mouse), and one or more mechanisms for providing results associated with the processing unit (for example, printer). The processor 16, memory 28, and input/output interface 22 can be interconnected, for example, via bus 18 as part of a data processing unit 12. Suitable interconnections, for example via bus 18, can also be provided to a network interface 20, such as a network card, which can be provided to interface with a computer network, and to a media interface, such as a diskette or CD-ROM drive, which can be provided to interface with suitable media.

Accordingly, computer software including instructions or code for performing the methodologies of the invention, as described herein, may be stored in one or more of the associated memory devices (for example, ROM, fixed or removable memory) and, when ready to be utilized, loaded in part or in whole (for example, into RAM) and implemented by a CPU. Such software could include, but is not limited to, firmware, resident software, microcode, and the like.

A data processing system suitable for storing and/or executing program code will include at least one processor 16 coupled directly or indirectly to memory elements 28 through a system bus 18. The memory elements can include local memory employed during actual implementation of the program code, bulk storage, and cache memories 32 which provide temporary storage of at least some program code in order to reduce the number of times code must be retrieved from bulk storage during implementation.

Input/output or I/O devices (including but not limited to keyboards, displays, pointing devices, and the like) can be coupled to the system either directly or through intervening I/O controllers.

Network adapters 20 may also be coupled to the system to enable the data processing system to become coupled to other data processing systems or remote printers or storage devices through intervening private or public networks. Modems, cable modem and Ethernet cards are just a few of the currently available types of network adapters.

As used herein, including the claims, a “server” includes a physical data processing system (for example, system 12 as shown in FIG. 5) running a server program. It will be understood that such a physical server may or may not include a display and keyboard.

It should be noted that any of the methods described herein can include an additional step of providing a system comprising distinct software modules embodied on a computer readable storage medium; the modules can include, for example, any or all of the appropriate elements depicted in the block diagrams and/or described herein; by way of example and not limitation, any one, some or all of the modules/blocks and or sub-modules/sub-blocks described. The method steps can then be carried out using the distinct software modules and/or sub-modules of the system, as described above, executing on one or more hardware processors such as 16. Further, a computer program product can include a computer-readable storage medium with code adapted to be implemented to carry out one or more method steps described herein, including the provision of the system with the distinct software modules.

One example of user interface that could be employed in some cases is hypertext markup language (HTML) code served out by a server or the like, to a browser of a computing device of a user. The HTML is parsed by the browser on the user's computing device to create a graphical user interface (GUI).

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method of operating a support system comprising:
enabling electronic communications with at least one senor of user conditions and at least one database of user activity;
collecting, via the electronic communications, a plurality of data points from the sensor and the database;
determining a context of the user;
calculating at least one affinity score of the user for each of at least one patient;
predicting a risk of passive illness associated with the user;
identifying, using the risk of passive illness, an amelioration action; and
outputting a signal reducing the risk of the passive illness.

2. The method of claim 1, wherein the context of the user includes a condition of the patient.

3. The method of claim 1, wherein affinity score includes at least one of a user-patient interaction, a user-medication interaction, a user-meal interaction, and a user-caregiver interaction.

4. The method of claim 1, wherein calculating the affinity score comprises:
detecting a plurality of interactions of the user;
detecting a time of each interaction;
detecting a proximity of the user and the patient at each interaction; and
predicting a relationship of user with the patient using the interactions, the times and the proximities.

5. The method of claim 1, wherein predicting the risk of passive illness further comprises:
detecting an emotional trajectory of the user;
detecting a cognitive trajectory of the user;
determining a health condition of the user; and
inferring the risk of passive illness from the emotional trajectory, the cognitive trajectory, and the health condition of the user.

6. The method of claim 1, further comprising:
estimating an impact of a calendared event in the one database of user activity of the risk; and
determining that the impact is greater than a threshold,
wherein the signal reducing the risk of the passive illness modifies one or more calendars of the user.

7. The method of claim 6, wherein the modification is one of removing the calendared event from the user calendar and placing an indicator on the calendared event reducing an importance of the calendared event.

8. The method of claim 1, wherein the database of user activity includes data for at least one of scheduled calls, scheduled travel, and scheduled appointments.

9. The method of claim 1, wherein the amelioration action includes at least one of a recommendation notification and a modification of the at least one database.

10. The method of claim 1, wherein the ameliorative action is identified using the risk of passive illness and a database of historic outcomes.

11. The method of claim 1, further comprising:
collecting feedback from the user given the amelioration action; and
storing the feedback into the database of historic outcomes.

12. The method of claim 1, further comprising building a health graph network comprising a plurality of nodes corresponding to individual users and edges corresponding to risk of passive illness calculated from the affinity score.

13. A non-transitory computer readable storage medium comprising computer executable instructions which when executed by a computer cause the computer to perform a method of operating a support system, the method comprising:
enabling electronic communications with at least one senor of user conditions and at least one database of user activity;
collecting, via the electronic communications, a plurality of data points from the sensor and the database;
determining a context of the user;
calculating at least one affinity score of the user for each of at least one patient;
predicting a risk of passive illness associated with the user;
identifying, using the risk of passive illness, an amelioration action; and
outputting a signal reducing the risk of the passive illness.

14. The non-transitory computer readable storage medium of claim 13, wherein calculating the affinity score comprises:
detecting a plurality of interactions of the user;
detecting a time of each interaction;
detecting a proximity of the user and the patient at each interaction; and
predicting a relationship of user with the patient using the interactions, the times and the proximities.

15. The non-transitory computer readable storage medium of claim 13, wherein predicting the risk of passive illness further comprises:
detecting an emotional trajectory of the user;
detecting a cognitive trajectory of the user;
determining a health condition of the user; and
inferring the risk of passive illness from the emotional trajectory, the cognitive trajectory, and the health condition of the user.

16. The non-transitory computer readable storage medium of claim 13, further comprising:
estimating an impact of a calendared event in the one database of user activity of the risk; and
determining that the impact is greater than a threshold,
wherein the signal reducing the risk of the passive illness modifies one or more calendars of the user.

17. The non-transitory computer readable storage medium of claim 16, wherein the modification is one of removing the calendared event from the user calendar and placing an indicator on the calendared event reducing an importance of the calendared event.

18. The non-transitory computer readable storage medium of claim 13, wherein the ameliorative action is identified using the risk of passive illness and a database of historic outcomes.

19. The non-transitory computer readable storage medium of claim 13, further comprising:
collecting feedback from the user given the amelioration action; and
storing the feedback into the database of historic outcomes.

20. The non-transitory computer readable storage medium of claim 13, further comprising building a health graph network comprising a plurality of nodes corresponding to individual users and edges corresponding to risk of passive illness calculated from the affinity score.
